(19) United States
(12) Patent Application Publication
(10) Pub. No.: US 2024/0050274 A1
(43) Pub. Date: Feb. 15, 2024

(54) PROXIMAL ULTRASONIC WATER PRESSURE DEVICE FOR ULTRAFINE GAUGE VITRECTOMY IN COMBINED SAMPLING AND DRUG DELIVERY DEVICE

(71) Applicant: TWENTY TWENTY THERAPEUTICS LLC, South San Francisco, CA (US)

(72) Inventors: Alexandre Tumlinson, South San Francisco, CA (US); Dimitri Azar, South San Francisco, CA (US)

(21) Appl. No.: 18/232,271

(22) Filed: Aug. 9, 2023

Related U.S. Application Data
(60) Provisional application No. 63/396,426, filed on Aug. 9, 2022.

(51) Int. Cl. A61F 9/007; A61M 1/00; A61F 9/00

(52) U.S. Cl. CPC A61F 9/00745; A61M 1/815; A61F 9/0017; A61M 2205/3306; A61M 2205/3379; A61M 2205/3331; A61M 2205/3368

(57) ABSTRACT

A device includes an eye penetration member extending from a proximal end to a distal end and including a body defining a lumen for receiving a liquid column and including at least one port extending to the lumen. The distal end is configured for insertion into the vitreous humor of an eye. A pressure modulation system is in fluid communication with the lumen and includes a low frequency modulation mechanism and a high frequency modulation mechanism that cooperate to draw vitreous humor into the lumen and liquefy the vitreous humor. The high frequency pressure modulation imparts acoustic pressure modulation to the liquid column at the proximal end of the eye penetration member which is transferred to the distal end of the eye penetration member.

### RELATED APPLICATIONS

This application claims the benefit of U.S. Provisional Application Ser. No. 63/396,426, filed Aug. 9, 2022, the entirety of which is incorporated herein by reference.

### TECHNICAL FIELD

The present invention relates to devices, systems, and methods for injection of substances into, and sampling of, aqueous and vitreous humors of the eye. The disclosed intravitreal injection and sampling device has particular but not exclusive utility for diagnosis and treatment of ophthalmic disorders in humans.

### BACKGROUND

Vitreous humor is a colorless, gelatinous fluid within an eye or eyeball of humans or other vertebrates composed of approximately 98-99% water with trace amounts of hyaluronic acid, glucose, anions, cations, ions, and a fine network of collagen. Vitreous humor provides support to the surrounding structures of the eye, absorbs mechanical trauma, and provides circulation and regulation of oxygen, metabolites and nutrients. It is produced largely by cells of the ciliary body. Changes in vitreous structure that occur with aging, are important in the pathogenesis of many vitreoretinal diseases.

Intraocular pressure (TOP) quantifies the pressure of the vitreous humor inside the eye. Many individuals suffer from disorders, such as glaucoma, that are associated with chronic heightened IOP. Over time, heightened IOP can cause damage to the optical nerve of the eye, leading to loss of vision.

Presently, treatment of ophthalmic disorders mainly involves periodically administering pharmaceutical agents to the eye. These drugs can be delivered by, for example, intravitreal injection. Intravitreal injection is one of the most common surgical procedures performed in ophthalmology today. A variety of drugs are delivered directly to the clear vitreous gel that supports the globe of the eye. These drugs act directly in the vitreous or in the surrounding retinal tissues over the following months. For example, intravitreal injection is a common route of delivery for vascular endothelial growth factor inhibiting (anti-VEGF) proteins, which are highly potent compounds tolerated at high doses, with intravitreal half-lives about one week. Anti-VEGF biologics and steroids are the most commonly administered drugs by this route. These drugs may be administered on a chronic basis.

One recommended procedure for intravitreal injection includes preparation of an injection needle, topical anesthesia and disinfection of the eye surface, holding the eye open with a lid speculum or other means, optional lateral dislocation of the conjunctiva at the injection site, and insertion of the needle a few mm lateral to the limbus to approximately the full depth of the needle, injecting the drug, withdrawing the needle, and allowing the conjunctiva to cover the injection site. Post injection care typically includes a basic verification of functional vision such as requesting the patient to count the number of fingers shown by the doctor. This functional test verifies that acute TOP increase due to injection has not impacted the optic nerve head in a way that requires immediate relief.

Another important ophthalmic procedure is vitreous sampling. Vitreous sampling may inform various aspects of eye care. Samples of vitreous may be analyzed for cellular content and extracellular structure by histology or immunologic analysis. Histology can, for example, provide a definitive diagnosis for the type of infection causing endophthalmitis.

Identification of the type of immune cells present and the immune mediator proteins expressed may inform the treatment of uveitis. Identification of the amount of VEGF present in the vitreous may give an indication of how likely imminent neovascularization is to occur or how likely it is that VEGF compounds are responsible for an observed case of neovascularization. Non-responders to anti-VEGF treatment remains one of the most troublesome aspects of treating neovascularization in exudative, age-related vascular degeneration (also known as wet AMD) and diabetic retinopathy.

Two common methods of vitreous sampling—with a cutter or with needle aspiration—appear to be approximately equivalent for the purposes of protein analysis. A state of the art miniature cutting tool may be delivered through a 23-gauge trocar. Needle aspiration may be performed with needles as small as 30-gauge (about half the diameter of 23 gauge). Fine gauge may increase the probability of a dry tap and/or change the properties of the aspirated material by acting as a filter. Small gauge may have an advantage in that traction may not be introduced on the gel matrix because the gel matrix cannot be pulled into the small needle bore. Vitreous samples are typically frozen or otherwise stabilized so that they can be processed in a laboratory outside of the operating room or ophthalmic office setting.

Injection of therapeutic doses of medication into the vitreous or aqueous humor inside the eye can increase TOP by as much as 25 mmHg, which is substantially greater than threshold levels that are considered potentially harmful. Evidence shows that while such TOP increases are transient, they are in fact associated with an iatrogenic glaucoma resulting in measurable loss of nerve fiber layer and visual function over a course of only several treatments in patients with ‘normal’ resting TOP. See Saxena, S., Lai, T. Y., Koizumi, H. et al., “Anterior chamber paracentesis during intravitreal injections in observational trials: effectiveness and safety and effects,” International Journal of Retina and Vitreous, 5, 8 (2019). Therefore, it is sometimes desirable to remove a small volume of humor (whether aqueous, vitreous or both) from the eye before injecting a comparable volume of medication. However, removal of a volume of humor may result in insufficient pressure, which can also be harmful to the eye.

Therefore, in the case of diagnostic sampling of humors, it may be necessary or beneficial to inject a volume of fluid (whether medicated or otherwise) to replace the withdrawn humors. In either case, care must be taken to ensure that the removed and injected volumes are comparable, and in either case, two separate procedures (a sampling procedure and an injection procedure) are typically required.

### SUMMARY

In one example, the present invention relates to a sampling and drug delivery device for liquefying and removing vitreous gel from the eye using a needle probe inserted into the eye. The remainder of the vitreous remains largely unaltered and the liquefied sample can be collected by an extraction mechanism. Energy used to drive the liquefaction process can include acoustic vibration, a mechanical guillotine cutter, a mechanical screw cutter, electrical energy, optical energy, chemical action, and/or enzymatic action.

The energy for driving the liquefaction is communicated by a column of liquid in the aspiration path of the needle probe. An approximately static low pressure is applied to the column of liquid to drive a bulk flow in the direction out of the eye through the needle probe. An ultrasonic modulation of the pressure is simultaneously applied at the proximal end of the needle. The pressure variation inside the needle is communicated to a distal tip, where at least one small port communicates the low and high frequency pressure to the outside vitreous. A small amount of vitreous gel is drawn into the small port by the combination of negative pressure and ultrasonic modulation. The vitreous gel oscillated in the small port is broken down into a liquid which can be aspirated into the column of liquid and further drawn out of the eye. Once a sample of vitreous tissue, e.g., about 20 to about 300 μL, is obtained, an approximately isovolumetric drug injection into the eye can performed.

Other objects and advantages and a fuller understanding of the invention will be had from the following detailed description and the accompanying drawings.

### BRIEF DESCRIPTION OF THE DRAWINGS

FIG. 1 is a schematic illustration of an example combined biological sampling and injection assembly in accordance with the present invention.

FIG. 2 is a section view of the assembly of FIG. 1 taken along line 2-2.

FIG. 3 is a schematic illustration of the assembly of FIG. 2 is use.

FIG. 4A is schematic illustration of another example combined biological sampling and injection assembly.

FIG. 4B is an enlarged view of a portion of FIG. 4A.

FIG. 5 is schematic illustration of another example combined biological sampling and injection assembly.

FIG. 6 is schematic illustration of another example combined biological sampling and injection assembly.

FIG. 7 is schematic illustration of another example combined biological sampling and injection assembly.

FIG. 8 is schematic illustration of another example combined biological sampling and injection assembly.

FIG. 9 is schematic illustration of a portion of another example combined biological sampling and injection assembly.

### DETAILED DESCRIPTION

The subject matter described herein relates to devices, systems, and methods for injection of substances into, and sampling of, aqueous and vitreous humors of the eye. The disclosed intravitreal injection and sampling device has particular but not exclusive utility for diagnosis and treatment of ophthalmic disorders in humans.

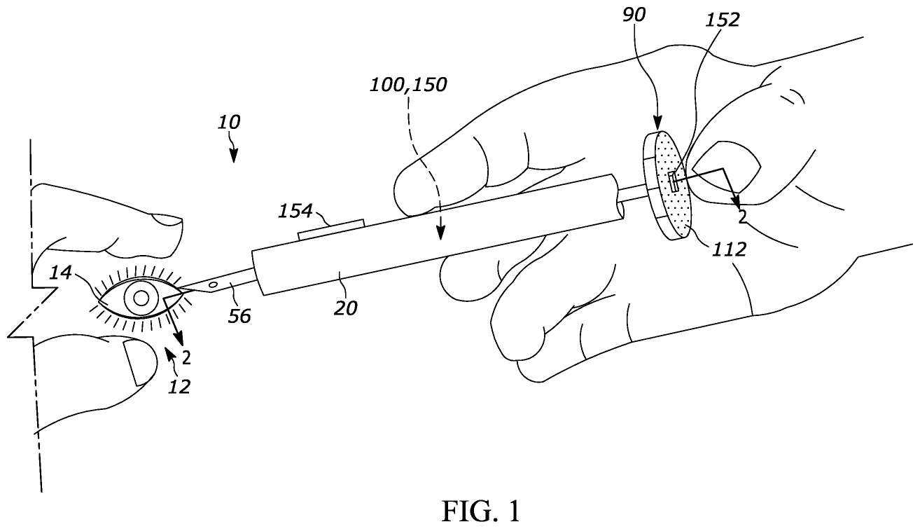

FIG. 1 illustrates an example combined biological sampling and injection assembly or device 10 in accordance with the present invention. The device 10 is operable by a user, e.g., a physician, to obtain a sample of vitreous humor 14 from an eye 12 of a patient.

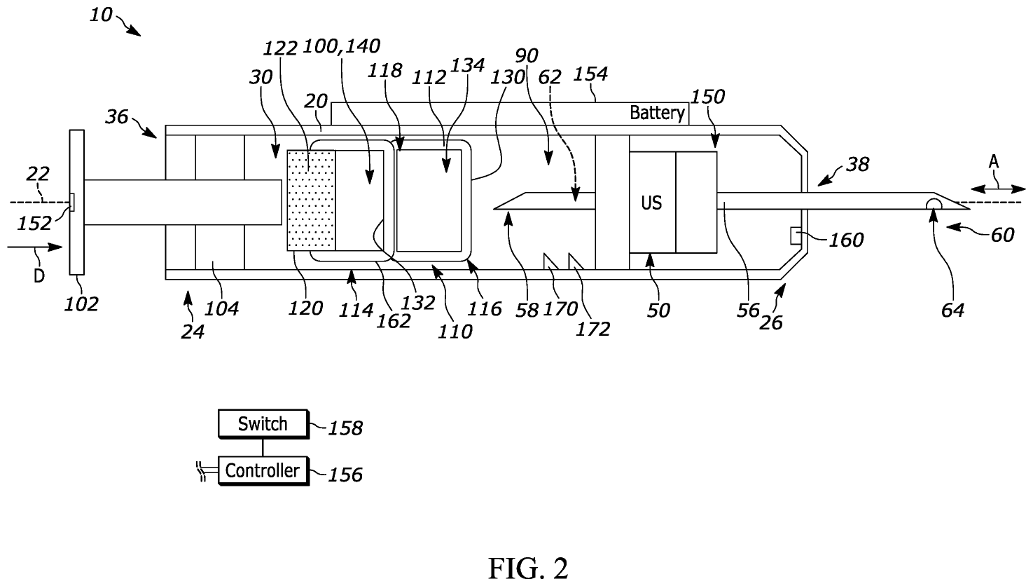

Referring to FIG. 2, the device 10 includes a tubular body 20 extending along a centerline 22 from a first or proximal end 24 to a second or distal end 26. A central passage 30 extends along the centerline 22 and along the entire length of the body 20 from a proximal opening 36 at the first end 24 to a distal opening 38 at the second end 26.

An eye penetration member or needle probe 50 is provided in the central passage 30. The probe 50 includes a needle 56 extending longitudinally from a proximal end 58 to a distal end 60. Each end 58, 60 can have a pointed, angled configuration. A lumen 62 extends between the ends 58, 60 along the length of the needle 56. At least one port 64 extends radially from the periphery of the needle 56 radially inward to the lumen 62. It will be appreciated, however, that the port 64 could alternatively or additionally extend longitudinally through the distal end 60 of the needle 56. In other words, the port 64 can be coextensive with the lumen 62 (not shown).

A pressure modulation system 90 is provided for modulating the pressure of any fluid delivered to or provided within the lumen 62 and/or modulating movement of the needle 56. The system 90 includes a low frequency modulation mechanism 100 and a high frequency modulation mechanism 150. The low frequency modulation system 100 is coupled to/operable with a handle 102 extending through the proximal opening 36 into the central passage 30 of the body 20. A piston 104 encircles the handle 102 and forms a sliding fit with the interior of the body 20 such that the low frequency modulation system 100 is axially movable within and relative to the central passage 20.

An evacuated chamber 110 is provided within the central passage 30 between the handle 102 and the proximal end 58 of the needle 56. The evacuated chamber 110 includes a tubular body 112 extending between first and second ends 114, 116 and defining a chamber 118. One end of the chamber 110 is closed by a drug container 120 having a drug, e.g., a therapeutic agent, disposed therein. A first septum 130 closes the opposing end of the chamber 110. A second septum 132 is provided between the drug container 120 and the first septum 130. A blister of priming fluid 134, e.g., water, is provided in the chamber 110 between the first and second septums 130, 132.

A sample receiving chamber 140 is defined between the drug container 110 and the second septum 132. The sample receiving chamber 140 is evacuated of air and thereby at vacuum pressure. That said, in one example the vacuum chamber 140 acts as the low frequency modulation mechanism 100.

The high frequency modulation mechanism 150 includes an actuator 152 provided in the handle 102 and connected to the needle 56. The high frequency modulation mechanism 150 is powered by a battery 154 coupled to the body 20. The actuator 152 is also connected to a controller 156. In one example, the actuator 152 is connected to a piezoelectric drive (not shown) configured to apply ultrasonic pressure to the needle 56 such that the entire needle 56 axially vibrates in the manner indicated generally at A in FIG. 2. The ultrasonic pressure can be, for example, from about 20 kHz to about 50 kHz.

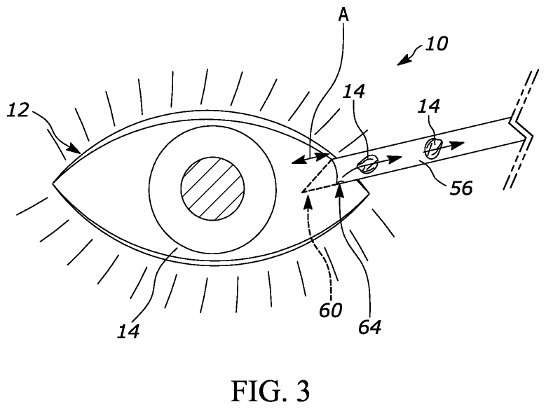

In operation and referring further to FIG. 3, the distal end 60 of the needle 56 is positioned adjacent the eye 12. The container 110 is advanced by pushing the handle 102 in the direction D towards the distal end 26 of the tubular body 20 until the proximal end 58 of the needle 56 pierces the first septum 130. This places the chamber 118 in fluid communication with the lumen 62 of the needle 56. When this occurs, the priming fluid 134 flows out of the chamber 118, through the lumen 62 of the needle 56, and out the distal end 60 thereof in order to purge the lumen of air, debris, etc. The priming fluid 134, however, still occupies the lumen 62 of the needle 56 and at least part of the priming chamber 118. That said, the priming fluid 134 forms/helps to define a liquid column that is continuous with the gel of the vitreous humor 14.

The now purged needle 56 is then inserted into the eye 12. It will be appreciated that a pressure or proximity sensor (not shown) can be provided on the tubular body 20 to help the user sense the position of the tubular body relative to the eye 12. Once the distal end 60 is positioned within the vitreous humor 14, the handle 102 is advanced further into the tubular body 20 in the direction D to advance the container 110 until the proximal tip 58 of the needle 56 pierces the second septum 132.

At this point, the high and low frequency modulation mechanisms 100, 150 can automatically cooperate to impart forces on the vitreous humor 14, including shear and viscous heating at the radial port 64, that act to disrupt the integrity of the vitreous humor. More specifically, the pressure modulation system 90 breaks apart and liquefies the vitreous humor 14 sufficient to draw portions of the vitreous humor into the radial port 64 of the needle 56 and into the liquid column, ultimately passing through the needle and into the sample receiving chamber 140.

In one example, piercing the second septum 132 with the proximal end 58 of the needle 56 places the vacuum of the chamber 140 in fluid communication with the liquid column, lumen 62, and vitreous humor 14. The low frequency, low pressure vacuum of the chamber 140 is lower than the mean pressure inside the eye 12 and, thus, the pressure differential draws vitreous humor 14 into the radial port 64 towards the sample receiving chamber. The user can modulate the low frequency vacuum by moving the handle 102 in a reciprocating, back-and-forth manner along the centerline 22, which thereby causes the drawn in vitreous humor 14 to move within and/or into and out of the lumen 62.

In this example shown, the vacuum pressure acts on the priming fluid 134 within the lumen 62. It will be appreciated, however, that the chamber 118 and priming fluid 134 therein can be omitted such that the vacuum pressure acts on air within the lumen 62. In either case, the vacuum pressure is capable of drawing the vitreous humor 14 into the radial port 64, whether the lumen 62 is filled with priming liquid 134 or empty. That said, the liquid column can be defined by the priming liquid 134 in the lumen 62 or the air (not shown) in the lumen.

At the same time, the actuator 152 can be activated by the user or automatically activated (in response to sensor readings) to begin high frequency modulation of the needle 56, thereby liquefying the back-and-forth moving vitreous humor 14 drawing into the radial port 64. More specifically, the actuator 152 activates the high frequency pressure modulation mechanism 150 to ultrasonically impart acoustic pressure modulation upon the proximal end 58 of the needle 56. The acoustic pressure modulation is transmitted through the liquid column from the proximal end 58 of the needle 56 to the distal end 60 to initiate the vitreous humor 14 liquefaction by creating a pressure gradient at the radial port 64. The acoustic pressure is therefore applied directly to the needle 56, causing the distal end 60 thereof to oscillate/vibrate longitudinally relative to the centerline 22 in the manner A.

Returning to FIG. 2, a series of latches 170, 172 can be provided along the interior of the tube 20 and extending radially towards the centerline 22. The latches 170, 172 are configured to sequentially limit the degree to which the container 110 can be advanced in the central passage 30 by operating the handle 102. Alternatively or additionally, the latches 170, 172 can limit the degree to which the container 110 advances automatically if a force is constantly applied to the handle 102, such as by a preloaded spring (not shown). In one example, a first latch 170 is provided distally from the container 110 to initially prevent piercing of the second septum 132 by the needle 56. Once the needle 56 has been evacuated by the priming fluid 134 and advanced into the vitreous humor 14, the controller 156 can radially retract the first latch 170 to allow the container 110 to be advanced distally until the needle 56 punctures the second septum 132 and establish fluid communication between the vitreous humor 14 and the sample receiving chamber 140 via the lumen 62.

One or more sensors can be provided on the device 10 for helping to control operation of the latches 170, 172. In one example, a proximity sensor or pad 160 is provided on the exterior of the tube 20 adjacent the distal opening 38. When the sensor 160 comes into contact with the sclera, a signal is sent from the sensor to the controller 156. In response to receiving the signal, the controller 156 automatically releases or retracts the first latch 170. With the first latch 170 retracted, the user can use the handle 102 to further advance the container 110 in the direction D towards the distal end 26 of the tube 20 until the needle 56 pierces the second septum 132. Once this occurs, the mechanical liquefaction of the vitreous humor 14 is automatically initiated.

An optical sensor 162 can be positioned on the container 110 and has a probe wavelength transmissive to the chamber 140 walls but opaque to the vitreous humor 14 sample entering the chamber. The optical sensor 162 measures the volume of the sample received by the chamber 140 and sends signals indicative thereof to the controller 156.

Alternative or additional sensors can be used to help determine and track the volume of vitreous humor 14 received by the chamber 142. This can include, for example, a pressure sensor coupled to the chamber 142 for measuring a pressure change therein to determine the volume of the vitreous fluid collected, a pressure sensor coupled to the chamber for measuring a pressure change therein to determine the volume of the vitreous fluid collected, and/or a temperature sensor coupled to the lumen 62 for measuring heat conduction therein to determine the volume of the vitreous fluid collected.

Once the chamber 140 is full, the system times out or the user aborts the sampling procedure, the controller 156 releases or retracts the second latch 172, thereby enabling further movement of the container 110 in the direction D by operation of the handle 102. The released second latch 172 allows the user to advance the container 110 with the handle 102 in the direction D until the needle 56 enters the drug container 120 and places the drug 122 in fluid communication with the eye 12 via the lumen 62. Thereafter advancing the plunger 102 in the direction D pushes the drug 122 out of the container 120 to be fully injected into the eye where the vitreous humor 14 sample was removed.

After the drug 122 is delivered, the handle 102 can be depressed further in the direction D, which automatically causes another latch (not shown) to urge the needle 56 back into the central passage 30 until the distal end 60 no longer extends out through the distal opening 38 of the tube 20. This retraction allows the needle 56 to be protected while the device 10 is removed from the eye 10.

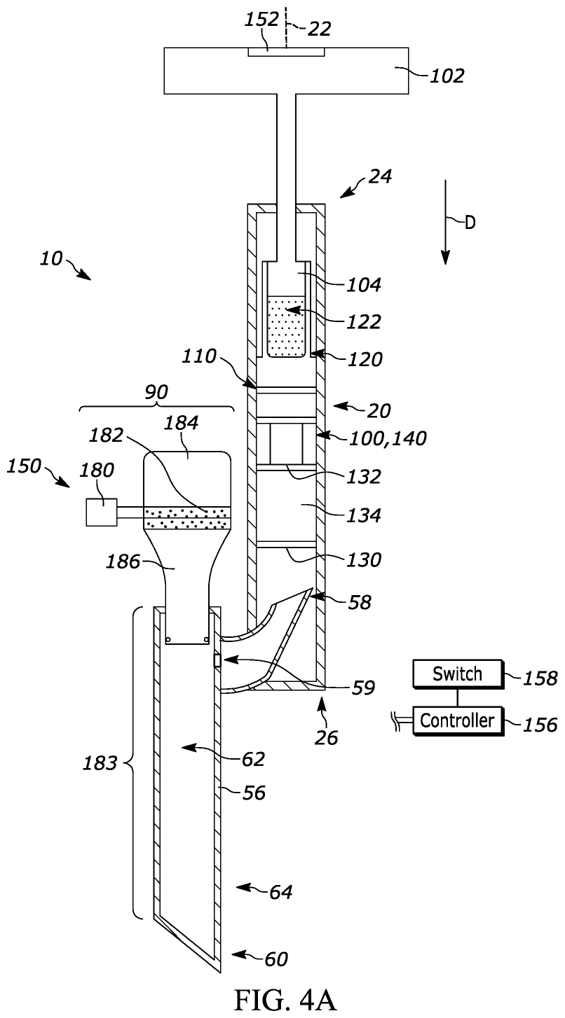

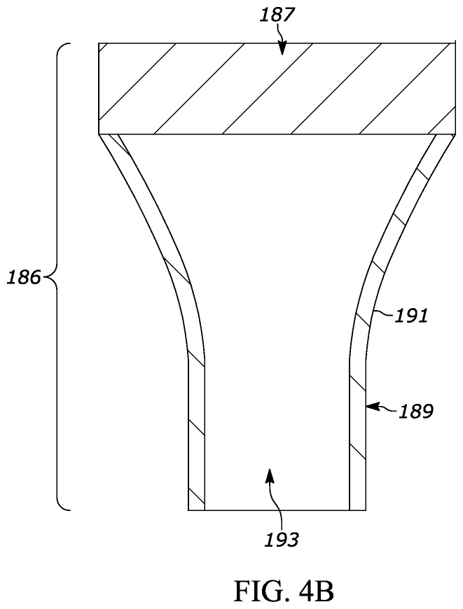

FIGS. 4A-4B illustrate a modified version of the assembly 10 of FIG. 2. Features in FIGS. 4A-4B that are similar to those in FIG. 2 carry the same reference numbers. Although some features from FIG. 2 are not illustrated in FIG. 4A, such as latches, sensors, etc., it will be appreciated that any of the features of the device 10 of FIG. 2 can be implemented into the device of FIG. 4A, and vice versa.

With that in mind, the high frequency modulation mechanism 150 of FIG. 4A includes a piezo drive 180 coupled to piezoelectric crystals 182 for energizing the same upon activation of the actuator 152 in the handle 102. The drive 180 can operate at about, for example, 20 kHz to about 50 kHz. A back mass 184 cooperates with the energized piezoelectric crystals 182 to vibrate an acoustic horn 186 connected to the proximal end 58 of the needle 56. The horn 186 can be solid and/or fluid in construction.

In one instance shown in FIG. 4B, the horn 186 includes a solid portion 187 and a liquid portion 189. The solid portion 187 is secured/connected to the back mass 184. The liquid portion 189 extends distally away from the solid portion 187 and into the lumen 62. The liquid portion 189 can include a tube 191 and a liquid 193, e.g., an aqueous liquid, disposed therein. The longitudinal cross-section of the horn 186 decreases in a direction extending from the back mass 184 towards the distal end 64 of the needle 56.

In operation, the handle 102 is advanced in the direction D to move the container 110 and thereby cause the proximal end 58 of the needle 56 to puncture chamber 118. This, in turn, causes the priming liquid 134 to form a liquid column 183 in the needle 56. Further advancing the handle 102 in the direction D causes the receiving chamber 140 to be placed in fluid communication with the lumen 62, thereby applying vacuum pressure to the lumen.

At the same time, the actuator 152 can be activated by the user or automatically activated (in response to sensor readings) to begin high frequency modulation of the needle 56, thereby liquefying the vitreous humor 14 drawing into the radial port 64. More specifically, the actuator 152 imparts acoustic pressure modulation on the liquid column 183 at the proximal end 58 of the needle 56 which, in turn, imparts acoustic pressure modulation to the distal end 60 of the needle to initiate the vitreous humor 14 liquefaction. A constriction 59 in the needle 56 helps to mitigate/prevent pressure resonance from passing to the tubular body 20.

The acoustic pressure is therefore applied directly to the liquid column 183 and subsequently transferred to the ends 58, 60 of the needle 56—not directly applied to the needle. That said, both the low pressure vacuum within the sample receiving chamber 140 and the ultrasonic acoustic pressure generated by the high frequency modulation system 150 work simultaneously and in concert to both draw in the vitreous humor 14 and liquefy the same for transport through the liquid column 183 and into the sample receiving chamber 140.

It will be appreciated that the radial port 64 has a diameter that is relatively small compared to the diameter of the liquid column 183. That said, the radial port 64 is configured to mitigate the loss in acoustic pressure as the modulation is performed. With this in mind, the narrowing cross-section of the horn 186 (and optionally narrowing of the needle body) helps amplify the pressure and/or velocity of the flow in the lumen. Moreover, the length of the waveguide (horn 186) supports an acoustic resonance of the high frequency modulation to maximize the pressure difference across the port 64 relative to the high frequency modulation created at the proximal end 58 of the needle 56.

Additionally, the length of the liquid column 183 and the frequency of the high frequency drive are configured to precisely tune the properties of the liquid column. The liquid column should be considered as an acoustically resonant structure. For example, the speed of sound in water is approximately 1480 m/s and if the driving frequency is 28.5 kHz, a half wave segment is approximately 26 mm and a quarter wave approximately 13 mm. For a half wave, cylindrical water column 183, the pressure and velocity at the distal end 60 of the needle 56 opposite the modulator should have a similar magnitude as the driving modulator and opposite phase. That is, using a half wave length tube, or multiples of half wavelengths, the pressure at the distal end 60 of the needle 56 acts as if the modulator were directly present at that location. Consequently, the length of the waveguide is dependent on an integer number of half wavelengths of the acoustic frequency in the liquid column 183.

Nodes and anti-nodes of vibration are reproduced at half wave intervals along the liquid column 183. Conversely, the conditions are opposite at quarter wave intervals, if a vibrational node exists at one point, a vibrational anti-node exists a quarter wavelength further on the path. This relationship can be used to impedance match the components; for example the piezo stack can generate a very large force over a very short distance at a node, whereas it is desirable to generate significant motion of the fluid at the walls of the port. The cross section of the fluid path can also be used to manipulate the magnitude of the wave motion as an acoustic horn. As the cross section narrows toward the port, the velocity and peak amplitude of the wave motion increases and, thus, the shape of the waveguide can provide for an acoustic pressure amplification. Some flexibility in the length of the eye penetration member may be achieved by adding cylindrical half wavelength sections to the design without dramatically altering other parts of the geometry.

For complex geometries, and to include boundary effects and interactions with solid materials, the geometric design may be further tuned beyond these basic principals using finite element simulation tools such as COMSOL multiphysics software. Because the speed of sound in the fluid will depend on the specific density and rheology of the fluid in the liquid column, the frequency of the high frequency drive may require dynamic tuning to maintain resonance of the system as the temperature changes or as material with properties different from the priming fluid is aspirated. Feedback for the dynamic frequency tuning may be derived from the electrical impedance of the piezo as it interacts with the resonant system.

In any case, vibration of the horn 186 imparts ultrasonic pressure modulation to the liquid column 183, which cooperates with the low pressure vacuum within the sample receiving chamber 140 to cause oscillation of the liquid column into and out of the radial port 64 in order to break up the vitreous humor 14 and draw it into the lumen 62 and, ultimately, into the sample receiving chamber 140. That said, vibrating the horn 186 transmits the acoustic pressure modulation through the liquid column from the proximal end 58 of the needle 56 to the distal end 60 to cause oscillation/vibration thereof.

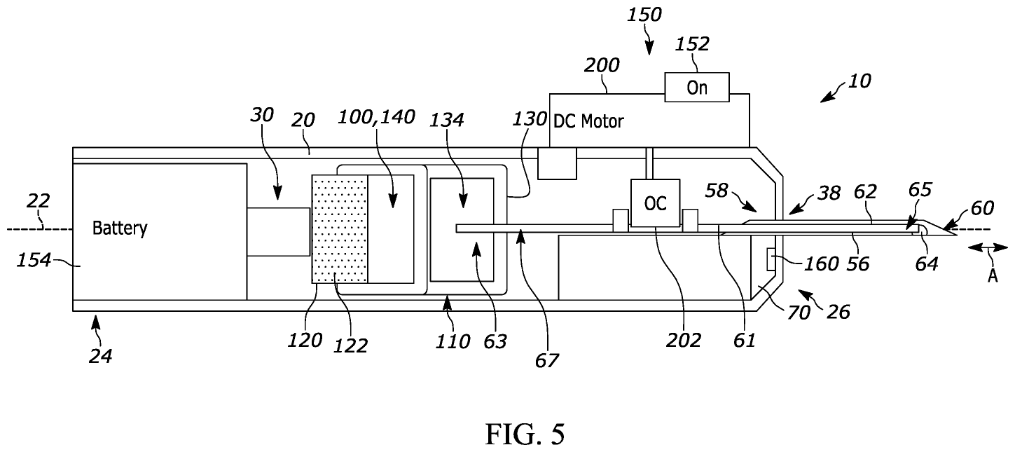

Alternatively configurations for the device 10 are illustrated in FIGS. 5-8. In these configurations, the low frequency vacuum pressure generated by the receiving chamber 140 is arranged in series with a mechanical modulation mechanism 150 that oscillates or reciprocates a tube 61 within the needle 56 in a back-and-forth manner along the centerline 22 in order to help draw the vitreous humor 14 into the radial port 64, through the liquid column, and into the receiving chamber.

In each configuration shown in FIGS. 5-8, the needle 56 is fixed to either the distal end 26 of the tubular body 20 or the projection 70, which is fixed to the interior wall of the tubular body. The tube 61 extends through the lumen 62 of the needle 56 and includes a first end 63 extending towards the container 110 (and piercing the same during operation) and a second end 65 terminating adjacent the port(s) 64 in the needle 56. A lumen 67 extends the entire length of the tube 61. As will be discussed, the tube 61 is axially movable relative to the stationary needle 56, receives the liquid column from the receiving chamber 140, and transfers the liquid column to the lumen 62 of the needle 56.

In FIG. 5, the mechanism 150 includes a DC motor 200 that drives an offset cam 202 secured to the tube 61 and powered by the battery 154. In operation, once the receiving chamber 140 is punctured by the first end 63 of the tube 61 and the low frequency pressure applied to the lumen 67, the user can press the actuator 152 to activate the motor 200. The motor 200 drives the offset cam 202 to cause the tube 61 to act as a guillotine cutter, i.e., move in an oscillating motion along the centerline 22 and relative to the stationary needle 56 as indicated generally at A. The vacuum from the receiving chamber 140 thereby cooperates with the oscillating tube 61 to remove and draw in vitreous humor 14 into the radial port 64 to ultimately reside in the sample receiving chamber 140.

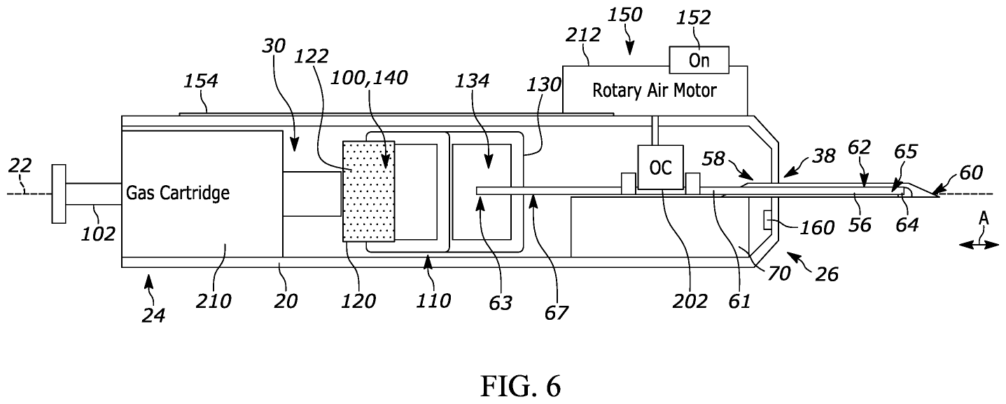

In FIG. 6, the mechanism 150 includes a rotary air motor 212 that drives the offset cam 202 secured to the tube 61. The rotary air motor 200 can be, for example, a turbine driven to rotate by a gas cartridge 210 provided in or on the tubular body 20. In operation, once the receiving chamber 140 is punctured by the first end 63 of the tube 61 and the low frequency pressure applied to the lumen 67, the user can press the actuator 152 to activate the motor 212. Gas from the gas cartridge 210 is delivered to the motor 212, which drives the offset cam 202 to cause the tube 61 to oscillate in the manner A relative to the stationary needle 56. The vacuum from the receiving chamber 140 thereby cooperates with the oscillating tube 61 to remove and draw in vitreous humor 14 into the radial port 64 to ultimately reside in the sample receiving chamber 140.

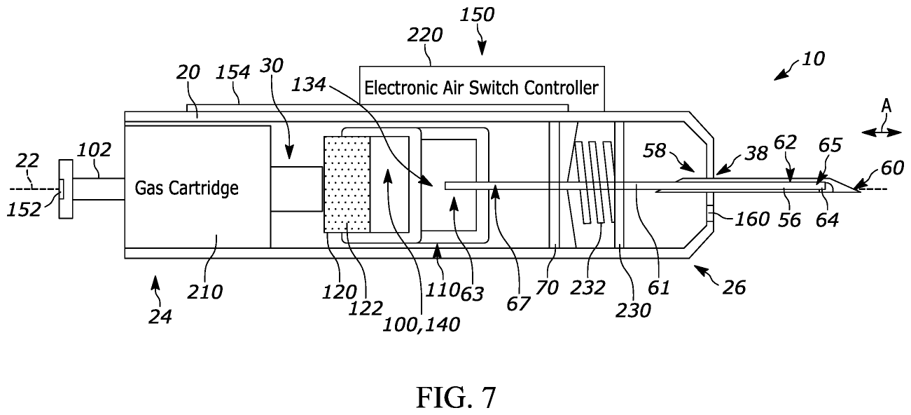

In FIG. 7, the mechanism 150 includes an electronic air switch controller 220 that drives a piston 230 secured to the tube 61. Consequently, the tube 61 is movable with the piston 230 relative to the stationary needle 56. A tension spring 232 is connected to the piston 230 and the stationary projection 70. In operation, once the receiving chamber 140 is punctured by the first end 63 of the tube 61 and the low frequency pressure applied to the lumen 67, the user can press the actuator 152 to activate the controller 220. Gas from the gas cartridge 210 is delivered to the controller 220, which relies on a valve system (not shown) to deliver air to the piston 230 in an intermittent, on-then-off manner.

Applying air to the piston 230 causes the piston—and the tube 61 secured thereto—to move towards the distal end 26 of the tubular body 26 against the bias of the tension spring 232. Turning off the air supply enables the tension spring 232 to automatically draw the piston 230 back towards the proximal end 24 of the tubular body 20. By rapidly alternating between supplying air to the piston 230 and shutting off the air supply, the air supply and tension spring 232 cooperate to move the tube 61 in the oscillating manner A relative to the stationary needle 56. The vacuum from the receiving chamber 140 thereby cooperates with the oscillating tube 61 to remove and draw in vitreous humor 14 into the radial port 64 to ultimately reside in the sample receiving chamber 140.

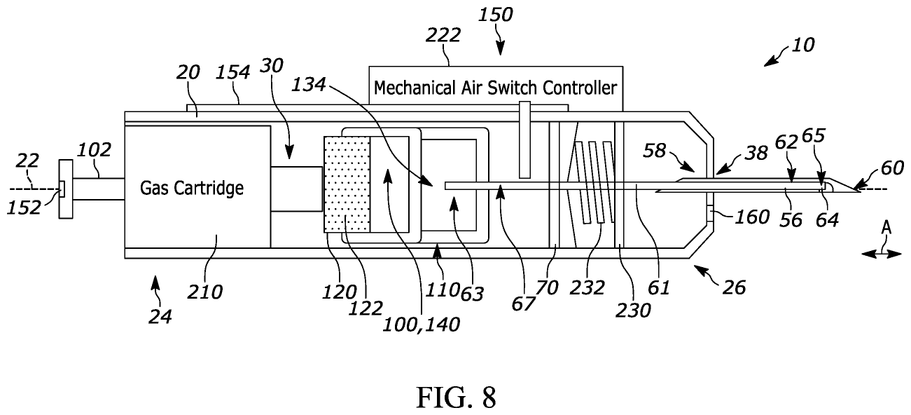

The mechanism in FIG. 8 is substantially similar to the mechanism 150 in FIG. 7, except that the electronic air switch controller 220 is replaced in FIG. 8 with a mechanical air switch controller 222.

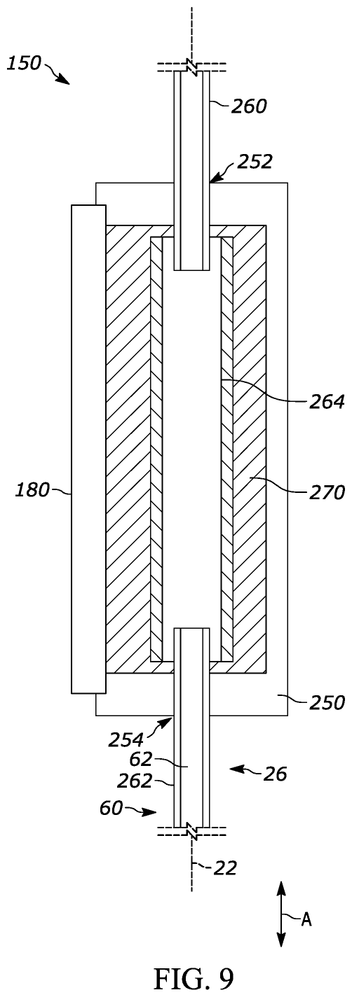

Another example of acoustic pressure modulation assembly 150 in shown in FIG. 9 and replaces the acoustic horn 186 in FIG. 4A. The assembly 150 includes a rigid pressure vessel 250. Openings 252, 254 are provided in opposing longitudinal ends of the vessel 250. The needle 26 in this example is formed from portions 260, 262 longitudinally aligned along the centerline 22. The first portion 260 extends through the opening 252 into the vessel 250. The second portion 262 extends through the opening 254 into the vessel 250. A flexible tube 264 is connected to each portion 260, 262 within the vessel 250 to provide fluid communication between the portions 260, 262. The tube 264 can be formed from, for example, kapton. A liquid 270 is provided within the vessel 250 and surrounds the flexible tube 264.

The piezo drive 180 (see FIG. 4A) is coupled to the pressure vessel 250 and energizes piezoelectric crystals (not shown) in the pressure vessel 250. When this occurs, ultrasonic vibration is communicated to the flexible tube 264, which transmits the pressure variation into the liquid column. Consequently, the modulation from the piezo drive 180 is added to any low frequency pressure modulation added elsewhere such as by piercing the receiving chamber 140 by the proximal portion of the needle 260.

It will be appreciated that additional, alternative configurations for the modulation mechanism can be implemented into the assemblies of the present invention. To this end, the modulation mechanisms can be mechanical-based and formed as a drill bit inside the needle or rely on oscillating guillotine blades driven by a variety of motor types.

The modulation mechanisms can be non-mechanical in nature. For instance, an energy delivery device can be provided on the distal end of the needle for helping to denature the vitreous gel in the eye. In one example, the energy delivery device includes one or more electrodes arranged circumferentially about the distal end of the needle and selectively energized by the controller. Furthermore, the electrodes can be arranged along the interior and/or exterior of the distal end of the needle.

Alternatively or additionally, one or more optical fibers can be provided on the distal end of the needle (along the interior and/or exterior thereof) for delivering a high energy pulse at a wavelength highly absorbed by water to locally disrupt the vitreous gel. The optical fibers can also be connected to and controlled by the controller.

Moreover, targeted Proteases such as collagenase, hyaluronidase, or others may be used to break down the specific proteins of the vitreous gel. These may be injected directly before the sampling or hours or weeks before sampling. Traditional chemicals such as strong acids or bases that are not particularly targeted may be injected alone or in combination with proteases to speed the chemical action to break down the structural proteins of vitreous.

The combined biological sampling and injection assemblies described herein are advantageous in that they provide for a small gauge, e.g., 30G or smaller, instrument that can directly penetrate the sclera, does not require protective measures for the sclera, does not require an external fluid source for the liquid column, and can be operated in a cordless manner. Furthermore, each of the assemblies provide multiple ways in which pressure modulation can be delivered to the liquid column at a manageable distance from the distal tip of the needle while performing the simultaneous operations of liquefying the vitreous sample as it is drawn in and out of the needle.

What have been described above are examples of the present invention. It is, of course, not possible to describe every conceivable combination of components or methodologies for purposes of describing the present invention, but one of ordinary skill in the art will recognize that many further combinations and permutations of the present invention are possible. Accordingly, the present invention is intended to embrace all such alterations, modifications and variations that fall within the spirit and scope of the appended claims.

What is claimed is:

1. A device comprising:
an eye penetration member extending from a proximal end to a distal end and including a body defining a lumen for receiving a liquid column and including at least one port extending to the lumen, the distal end being configured for insertion into the vitreous humor of an eye; and
a pressure modulation system in fluid communication with the lumen and including a low frequency modulation mechanism and a high frequency modulation mechanism that cooperate to draw vitreous humor into the lumen and liquefy the vitreous humor, wherein the high frequency pressure modulation imparts acoustic pressure modulation to the liquid column at the proximal end of the eye penetration member which is transferred to the distal end of the eye penetration member.

2. The device recited in claim 1, wherein the low frequency modulation unit comprises an evacuated container configured to apply a vacuum to the lumen for drawing vitreous humor through the at least one port into the lumen, the high frequency modulation mechanism being configured to apply acoustic pressure to the lumen to liquefy the vitreous humor.

3. The device recited in claim 1, wherein the low and high frequency modulation mechanisms are operated in a manner that causes the vitreous humor to flow into and out of the lumen until the vitreous humor is liquefied.

4. The device recited in claim 3, wherein the vitreous humor flows into and out of the lumen through the at least one port.

5. The device recited in claim 1, wherein the high frequency modulation mechanism is ultrasonic and operates at a frequency of about 22 kHz to about 50 kHz.

6. The device recited in claim 1, wherein the high frequency modulation mechanism comprises a piezo drive that energizes piezoelectric crystals to cause a solid acoustic horn to vibrate.

7. The device recited in claim 1, wherein the high frequency modulation mechanism comprises a piezo drive that energizes piezoelectric crystals to cause a fluid acoustic horn to vibrate.

8. The device recited in claim 7, further comprising a plunger slidably received in the eye penetration member and longitudinally displaceable by the user to move an evacuated chamber into engagement with the body for applying low frequency pressure to the lumen.

9. The device of claim 8, further comprising:
a liquid reservoir for holding a priming liquid for forming the liquid column prior to use of the device and to release the liquid column into the lumen when the plunger is moved distally to a first position; and
a vacuum and sample container configured to create a negative pressure within the lumen to finish the aspiration of the liquefied vitreous and to collect a sample of the liquefied vitreous when the plunger is moved to a second position distal of the first position.

10. The device recited in claim 9, further comprising a reservoir configured to release a therapeutic agent into the lumen for injection into the vitreous humor when the plunger is moved to a third position distal of the second position.

11. The device recited in claim 9, further comprising an optical sensor coupled to the vacuum and sample chamber for measuring the optical absorption of the vitreous fluid therein to determine the volume of the vitreous fluid collected.

12. The device recited in claim 9, further comprising a temperature sensor coupled to the vacuum and sample chamber for measuring a temperature change therein to determine the volume of the vitreous fluid collected.

13. The device recited in claim 9, further comprising a pressure sensor coupled to the vacuum and sample chamber for measuring a pressure change therein to determine the volume of the vitreous fluid collected.

14. The device of claim 1, further comprising a temperature sensor coupled to the lumen for measuring heat conduction therein to determine the volume of the vitreous fluid collected.

15. The device recited in claim 1, wherein the low frequency modulation mechanism comprises an evacuated tube.

16. The device recited in claim 1, wherein the low frequency modulation mechanism comprises a piston actuator.

17. The device of claim 1, wherein the liquid column transmits the acoustic pressure modulation from the proximal end of the eye penetration member to the distal end of the eye penetration member.

18. The device of claim 1, further comprising a controller configured to control the high frequency modulation mechanism for stopping and starting the supply of acoustic pressure to the lumen.

19. The device of claim 18, wherein the controller uses one or more inputs to determine when to start and/or stop liquefaction of the vitreous, wherein the one or more inputs includes at least one of a position of the eye penetration member in the eye, a position of internal components of the device, sensor data, a volume of vitreous sample acquired, and/or operator inputs.

20. The device of claim 1, further comprising:
a tubular body for receiving the eye penetration member; and
pressure sensor provided on the body and configured to sense the position of the body relative to the eye.

21. The device of claim 1, wherein the liquid column communicates liquid pressure between the eye and the pressure modulation system, wherein the pressure modulation system reduces the mean pressure within the liquid column below the mean pressure inside the eye to create negative pressure to pull the liquefied vitreous into the liquid column.

22. The device of claim 1 wherein the eye penetration member is configured to be a constant length waveguide for a driving frequency of the high frequency modulation mechanism for the liquid in the liquid column.

23. The device of claim 22, wherein the length of the waveguide supports an acoustic resonance of the high frequency modulation to maximize the pressure difference across the port relative to high frequency modulation created at the proximal end.

24. The device of claim 23, wherein the length of the waveguide is dependent on an integer number of half wavelengths of the acoustic frequency in the liquid medium.

25. The device of claim 23, wherein the shape of the waveguide provides for an acoustic pressure amplification.

26. The device of claim 23, wherein the cross sectional area of the liquid column at the at least one port is substantially smaller than the cross sectional area of the liquid column at the high frequency modulation.

27. The device of claim 1, wherein the at least one port is configured to liquefy the vitreous by disrupting the integrity of the vitreous gel using at least one of shear force or viscous heat.

28. A device comprising:
an eye penetration member extending from a proximal end to a distal end and including a body defining a lumen and including at least one port extending to the lumen, the distal end being configured for insertion into the vitreous humor of an eye;
a tube provided within the lumen and having a central passage; and
a pressure modulation system in fluid communication with the central passage and including a first, low frequency modulation mechanism and a second modulation mechanism that cooperate to draw vitreous humor into the lumen of the eye penetration member and liquefy the vitreous humor, wherein the low frequency modulation mechanism comprises a vacuum applied to the central passage and the second modulation mechanism longitudinally oscillates the tube relative to the eye penetration member.

29. The device of claim 28, wherein the second modulation mechanism comprises one of an electronic air switch controller, a mechanical air switch controller, a rotary air motor or a DC motor driving an offset cam for longitudinally oscillating the tube.

30. A method comprising:
inserting a sharpened distal end of an eye penetration member of a device into a globe of an eye, wherein the device comprises:
the eye penetration member having a proximal end in communication with a pressure modulation system and a body including a lumen for receiving a liquid column, wherein the sharpened distal end comprises at least one port, and
the pressure modulation system comprising a high frequency modulation mechanism and a low frequency modulation mechanism, wherein the high frequency modulation mechanism comprises an ultrasonic driver and the low frequency modulation mechanism comprises a container enclosing an evacuated space and movable by a plunger; and
liquefying a portion of a vitreous of the eye in contact with the sharpened distal end by applying ultrasonic pressure to the liquid column while the evacuated space is in fluid communication with the sharpened distal end of the eye penetration member to disrupt the integrity of the vitreous and thereby enable aspiration of at least a portion of the liquefied vitreous into the lumen of the eye penetration member through the at least one port.

31. The method of claim 30, further comprising:
priming the device by depressing the plunger to a first position to release liquid from a liquid reservoir of the device into the lumen to form the liquid column.

32. The method of claim 30, further comprising:
sampling the liquefied vitreous by depressing the plunger to a second position to open the container having the evacuated space to apply negative pressure to the lumen and thereby finish aspirating the portion of the liquefied vitreous into the lumen and collect a sample of the liquefied vitreous within the evacuated container.

33. The method of claim 30, further comprising:
injecting a therapeutic agent into the globe of the eye by depressing the plunger to a third position that releases the therapeutic agent from a therapeutic agent reservoir of the device into the lumen of the eye penetration member, wherein the therapeutic agent is injected through the at least one port.

34. The method of claim 30, wherein the inserting the sharpened distal end of the eye penetration member of a device into a globe of an eye further comprises inserting the sharpened distal end into at least one of the sclera, the pars plana, or the vitreous body of the eye.

35. The method of claim 30, wherein the liquefying the portion of the vitreous further comprises:
determining, by a controller of the device, when to start and/or stop liquefaction of the vitreous, wherein the one or more inputs includes at least one of a position of the eye penetration member in the eye, a position of internal components of the device, sensor data, a volume of vitreous sample acquired, and/or operator inputs.

36. The method of claim 30, wherein the ultrasonic driver comprises a piezo drive that energize piezoelectric crystals to vibrate an acoustic horn to apply ultrasonic pressure to the proximal end of the eye penetration member.

37. The method of claim 30, wherein the aspirating the portion of the liquefied vitreous further comprises reducing the mean pressure within the liquid column below the mean pressure inside the globe of the eye by applying ultrasonic pressure to the liquid column to create a negative pressure that pulls the portion of the liquefied vitreous into the liquid column.
